(19) United States
(12) Patent Application Publication
(10) Pub. No.: US 2006/0047264 A1
(43) Pub. Date: Mar. 2, 2006

(54) Endoscope flexible tube and method of manufacturing the tube

(75) Inventors: Atsushi Utsumi, Hyogo (JP); Tsunenori Arai, Tokyo (JP); Hiroshi Nagata, Tokyo (JP); Hunihiko Miyagi, Saitama (JP)

Correspondence Address: OSHA LIANG L.L.P., 1221 MCKINNEY STREET, SUITE 2800, HOUSTON, TX, 77010, US

(73) Assignees: Machida Endoscope Co., Ltd., Tokyo (JP); Keio University, Tokyo (JP)

(21) Appl. No.: 10/536,335

(22) PCT Filed: Dec. 2, 2003
(86) PCT No.: PCT/JP03/15384

§ 371 Date: May 25, 2005

(30) Foreign Application Priority Data
Dec. 2, 2002 (JP) 2002-349854

(51) Int. Cl. A61M 25/00

(52) U.S. Cl. 604/527

(57) ABSTRACT

A fluororesin tube body (10) of an endoscope flexible tube (1) is small in diameter and thin in wall thickness to such a degree that it is difficult to maintain its cross-sectional shape by itself when the flexible tube (1) is bent to the limit. A braid (20) woven from a plurality of flat bands (21) to form a tubular shape is disposed around the tube body (10). The mesh openings of the braid (20) are filled with filling adhesive (30), which bonds the tube body (10) and the braid (20) together. The filling adhesive (30) has enough elasticity to allow deformation of the braid (20) during the bending operation and enough adhesive strength to maintain the bond between the tube body (10) and the braid (20) even when the filling adhesive (30) is elastically deformed. Consequently, the braid (20) restricts the cross-sectional deformation of the tube body (10). Thus, the limit curvature of the flexible tube (1) can be increased.

### FIELD OF THE INVENTION

The present invention relates to a flexible tube which is particularly useful as a sheath of an insert portion of an endoscope or a catheter, or as a working channel or a lumen extending therethrough, and a method for manufacturing the same.

### DESCRIPTION OF THE RELATED ART

A flexible tube used in an endoscope or a catheter typically comprises a tube body made of resin such as polytetrafluoroethylene, whose outer circumferential surface is covered with a braid. The braid is woven from a plurality of strands to form a tubular shape. Bands made of resin (disclosed in Patent Reference 1 given below) and bands made of a plurality of metal fine wires arranged side-by-side to collectively define a flat band (disclosed in Patent Reference 2 given below) are well-known examples of such strands. In such a flexible tube, when a tensile force is applied to the tube, the braid stretches and reduces in diameter, thereby constricting the tube body tightly, and thus resisting the tensile force. It is also known that braids resist torsion very well.
Patent Reference 1: Japanese Patent Application Laid-Open No. H6-319686 (FIG. 4 on page 3)
Patent Reference 2: Japanese Patent Application Laid-Open No. H5-95893 (FIG. 1 on page 1)

Recently, demand for an endoscope that can be inserted and used in a small-diameter body cavity such as a human pancreatic duct has increased. Such an endoscope should be small in diameter and thin in wall thickness. For example, the diameter should be as small as around 2 mm in a flexible tube intended for use as a sheath of the insert portion and as small as around 0.5 mm in a flexible tube intended for use as a working channel extending therethrough. At the same time, the tube wall thickness should be as thin as several 10 μm. On the other hand, the tube body made of resin tends to be flattened when it is bent if its diameter is as small as and wall thickness is as thin as mentioned above. The circular cross-section of the tube body may be flattened and the tube body may be cracked, and may eventually fail. Therefore, allowable bending angle is limited in such tubes, in other words, the curvature is limited.

### BRIEF DESCRIPTION OF THE INVENTION

The present invention has been accomplished in order to solve the above-mentioned problem. According to the present invention, there is provided a flexible tube for an endoscope or a catheter comprising: a tube body being small in diameter and thin in wall-thickness to such a degree that it is difficult to maintain its cross-sectional shape by itself when the flexible tube is bent to the limit; a braid woven from a plurality of strands to form a tubular shape and disposed around the tube body; and filling adhesive filling the mesh openings of the braid, bonding the tube body and the braid together, the filling adhesive having enough elasticity to allow deformation of the braid (the variation in gap and angle between the strands) during the bending of the tube body, the filling adhesive having enough adhesive strength to maintain the bond between the tube body and the braid, thereby the braid restricts deformation of the cross-sectional shape of the tube body.

Owing to the features described above, the tube body can maintain its original cross-sectional shape even when it is bent to an angle at which it might be flattened and eventually fail if used alone. Consequently, the limit curvature of the flexible tube can be increased.

Preferably, the tube body is made of fluororesin, preferably of polytetrafluoroethylene (PTFE), and the strands woven into the braid are made of metal, preferably of stainless steel, and the filling adhesive is made from epoxy resin or urethane resin. Preferably, said filling adhesive has a tensile strength of from 400 to 1000 kgf/cm², an elongation of from 2 to 100%, and a tensile modulus of elasticity of from 10000 to 50000 kgf/cm². By using such materials, it is possible to form an extremely thin and small-diameter flexible tube having a desired elasticity, adhesive strength and bend resistance.

Preferably, the flexible tube is manufactured first by baking resin which is to define the tube body onto an outer circumference of a metal base material, which is thin linear in shape. The resin tube body formed in this way is then treated with an adhesion enhancing agent on the outer circumferential surface. Then the braid is disposed around the resin tube body. Next, the filling adhesive is applied plentifully to the mesh openings of the braid to bond the tube body and the braid together. After they are bonded together, the base material is pulled thinner and then removed out of the tube body. The flexible tube can be manufactured easily by the process described above.

### BRIEF DESCRIPTION OF THE DRAWINGS

FIG. 1 is a plan view of an embodiment of a flexible tube in accordance with the present invention.

FIG. 2 is a cross-sectional view of the flexible tube illustrated in FIG. 1.

### DETAILED DESCRIPTION OF THE INVENTION

One preferred embodiment of the present invention will be described hereinafter with reference to the accompanying drawings.

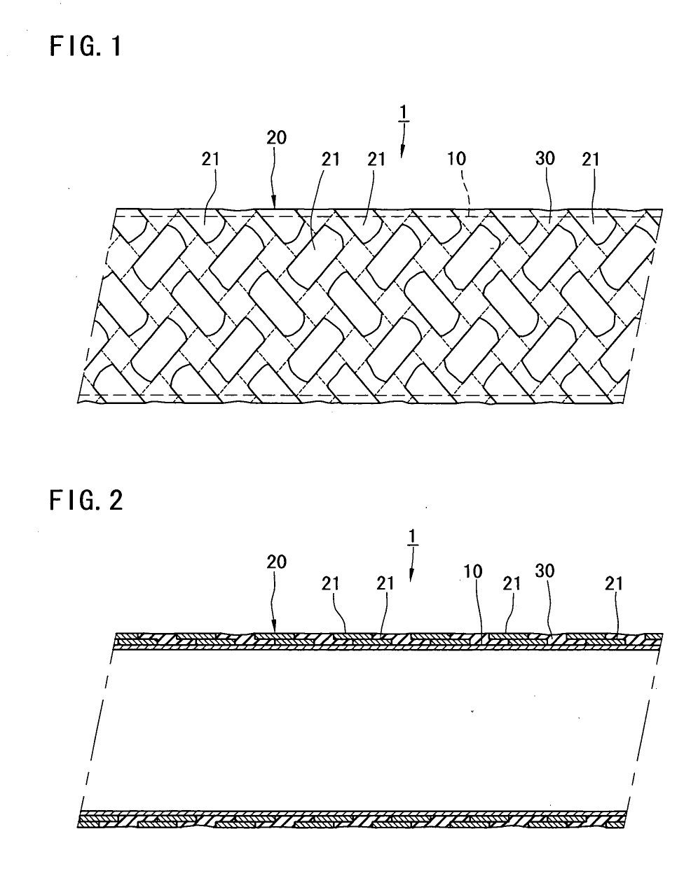

FIGS. 1 and 2 illustrate a flexible tube 1 intended for use as a working channel of an endoscope. An insert portion of the endoscope (not shown) has a sufficiently small diameter, approximately 2 mm to be inserted into a human pancreatic duct. The flexible tube 1 extends from a biopsy channel entry port of a body of the endoscope through the insert portion, then through a bendable portion to a distal end surface of a tip component of the endo scope.

The flexible tube 1 comprises a tube body 10 and a braid 20 disposed around the tube body 10. The tube body 10, which is made of fluoro resin such as polytetrafluoroethylene, is a small-diameter tube with a thin wall which extends in length. It has a circular cross-section with inner diameter of 0.5 mm and wall thickness of 10 μm.

The braid 20 is composed of a plurality of flat bands 21 made of metal such as stainless steel. The flat bands 21 are woven into tubular shape, which is then disposed around the tube body 10. The width of the flat band 21 is 50 μm and the thickness of the flat band 21 is 14 μm.

Therefore, the diameter of the flexible tube 1 is approximately 0.5 mm and the wall thickness of the flexible tube 1 is approximately 40 μm.

The mesh openings of the braid 20 are filled with filling adhesive 30, which bonds the tube body 10 and the braid 20 together. The filling adhesive 30 may be two-component epoxy resin adhesive made by mixing an equal amount of base resin which is primarily comprising bisphenol A epoxy resin and hardening agent which is primarily comprising polythiol. The viscosity of bisphenol A epoxy resin is approximately 50000 cp at 25° C. and the specific gravity of bisphenol A epoxy resin is approximately 1.17. The viscosity of polythiol is approximately 50000 cp at 25° C. and the specific gravity of polythiol is approximately 1.15.

The present inventors have tried various kinds of adhesive agents to find that adhesive agents comprising the above-mentioned components and having physical strength mentioned below (JIS K6911) are the most effective.

| | |
|---|---|
| Tensile strength: | 500 kgf/cm² |
| Elongation: | 3% |
| Tensile modulus of elasticity: | 22000 kgf/cm² |
| Compression strength: | 800 kgf/cm² |

Filling adhesive 30 comprising the above-mentioned components and having above-mentioned physical strength retains elasticity even after drying, and can sufficiently accommodate deformation of the braid 20 which may occur during the bending of the flexible tube 1. Furthermore, such filling adhesive, having high adhesion strength, can sufficiently maintain the bond between the tube body 10 and the braid 20 even when the adhesive itself was elastically deformed during the deformation of the braid 20.

Outer surface of the tube body 10 is adhesion-enhancement treated with an adhesion enhancing agent such as Tetra-Etch™ to enhance adhesion of the filling adhesive 30. In addition, the surfaces of the tube body 10 and the braid 20 are coated with primer (not shown) to facilitate adhesion of the filling adhesive 30.

A process for manufacturing a flexible tube 1 having the above described composition will now be described.

A copper wire with the diameter of 0.5 mm is used as a metal base material. First, Polytetrafluoroethylene resin is baked onto the outer circumferential surface of the copper wire to form the tube body 10. Adhesion enhancing agent such as Tetra-Etch™ is applied on the outer surface of the tube body 10 to adhesion activate the outer surface. In a separate process, stainless flat bands 21 are woven into a braid 20. The braid 20 is disposed around the tube body 10 made of the baked resin as described above. Then, primer is applied on the tube body 10 and the braid 20. Adhesive 30 is applied after the primer has dried. After the filling adhesive 30 has dried, the copper wire is pulled at both ends. This makes the copper wire elongate and reduce in diameter, and then come off the inner circumferential surface of the tube body 10. The copper wire is then removed out of the tube body 10, and the flexible tube 1 is formed.

The elastic filling adhesive 30 allows the braid 20 to deform by allowing the bands 21 to vary in distance and angle from each other during the bending operation of the flexible tube 1. This allows the flexible tube 1 to bend freely. The filling adhesive 30, on its own part, maintains its adhesion and thus maintains the bond between the tube body 10 and the braid 20 even when the adhesive 30 itself was elastically deformed during the bending operation of the flexible tube 1 as described above. Consequently, the braid 20 restricts deformation in cross-section at the bent portion of the tube body 10. Thus, the cross-section of the flexible tube 1 is maintained in a circular shape even when the tube body 10 is bent to the limit curvature at which it might be flattened and eventually fail if used alone. Consequently, the limit curvature of the flexible tube 1 can be increased.

Experiments by the present inventors have shown that a flexible tube according to this embodiment using filling adhesive with above-mentioned physical strength can be easily bent to a curvature radius of up to approximately 10 mm. On the other hand, when filling adhesive with a tensile strength of less than 400 kgf/cm², elongation of more than 100% and a tensile modulus of elasticity of less than 10000 kgf/cm² was used, the cross-section of the tube body was flattened when it was bent to a curvature radius of approximately 20 mm. In another instance, when filling adhesive having a tensile strength of more than 1000 kgf/cm², elongation of less than 2% and a tensile modulus of elasticity of more than 50000 kgf/cm² was used, the tube body failed by buckling when it was bent to a curvature radius of approximately 20 mm.

It should be noted that the present invention is not limited to the above embodiment but that many changes and modifications can be made thereto without departing from the spirit or scope of the present invention.

For example, the present invention is applicable to a sheath of an insert portion or a bendable portion of an endoscope. It is also applicable to a sheath of an insert portion or a bendable portion or a lumen of a catheter. A protective tube should be disposed around the braid when an flexible tube according to the present invention is used as a sheath of an endoscope or a catheter for medical use.

### INDUSTRIAL APPLICABILITY

The present invention is applicable to a flexible tube, especially of an endo scope.

What is claimed is:

1. A flexible tube for an endoscope or a catheter comprising:
a tube body being small in diameter and thin in wall-thickness to such a degree that it is difficult to maintain its cross-sectional shape by itself when said flexible tube is bent to the limit;
a braid woven from a plurality of strands to form a tubular shape and disposed around said tube body;
and filling adhesive filling the mesh openings of said braid, bonding said tube body and said braid together, said filling adhesive having enough elasticity to allow deformation of said braid during the bending of said tube body, said filling adhesive having enough adhesive strength to maintain the bond between said tube body and said braid even at the time of the elastic deformation of said filling adhesive, thereby said braid restrains deformation of the cross-sectional shape of said tube body.

2. A flexible tube according to claim 1, wherein said tube body is made of fluororesin, said strands composing said braid are made of metal and said filling adhesive is made from epoxy resin or urethane resin.

3. A flexible tube according to claim 1, wherein said filling adhesive has a tensile strength of from 400 to 1000 kgf/cm², an elongation of from 2 to 100%, and a tensile modulus of elasticity of from 10000 to 50000 kgf/cm².

4. A method for manufacturing a flexible tube according to claim 1, the method comprising the steps of: baking resin onto an outer circumferential surface of a thin linear metal base material to form said tube body, covering said resin tube body with said braid, bonding said tube body and said braid together by filling the mesh openings of said braid with said filling adhesive, pulling said base material to reduce its diameter, and removing said base material out of said tube body.
